(19) United States
(12) Patent Application Publication
(10) Pub. No.: US 2022/0111181 A1
(43) Pub. Date: Apr. 14, 2022

(54) INTRODUCER SHEATH HAVING A DISPLACEMENT SENSOR

(71) Applicant: Becton, Dickinson and Company, Franklin Lakes, NJ (US)

(72) Inventor: Casey Hebert, Loveland, CO (US)

(21) Appl. No.: 17/492,361

(22) Filed: Oct. 1, 2021

Related U.S. Application Data
(60) Provisional application No. 63/090,572, filed on Oct. 12, 2020.

(51) Int. Cl. A61M 25/06; A61M 25/00; A61M 25/09

(52) U.S. Cl. CPC A61M 25/0662; A61M 25/0097; A61M 25/09; A61M 2025/0681; A61M 2205/3327; A61M 2025/0042; A61M 2205/502; A61M 2205/3306; A61M 2205/18; A61M 2205/3592; A61M 2205/3317

(57) ABSTRACT

An introducer sheath may include a displacement sensor that detects displacement of a catheter within the introducer sheath. The displacement sensor may employ induction-based, optical-based or mechanical-based techniques for detecting the displacement. The displacement sensor may include a display on which the detected displacement is presented to a clinician. The displacement sensor may allow the clinician to provide user input to reset the displacement.

### RELATED APPLICATIONS

This application claims benefit of U.S. Provisional Patent Application No. 63/090,572, filed on Oct. 12, 2020, entitled INTRODUCER SHEATH HAVING A DISPLACEMENT SENSOR, which is incorporated herein in its entirety.

### BACKGROUND

An introducer sheath is a component of various vascular access systems. An introducer sheath is oftentimes used to introduce a catheter into a patient's vasculature. For example, in the Seldinger technique, a sharp, hollow needle is first used to puncture the vasculature. A guidewire may then be inserted into the vasculature via the lumen of the needle. The needle may then be withdrawn leaving the guidewire positioned in the vasculature. An introducer sheath, which may include a dilator, may then be passed over the guidewire and into the vasculature. With the introducer sheath positioned in the vasculature, the guidewire may then be withdrawn. The introducer sheath is typically retained in this position so that it may be used to introduce catheters or other devices into the patient's vasculature to perform a procedure such as angioplasty, stenting, thermoablation, embolization, biopsy, etc.

FIG. 1 provides one example of an introducer sheath assembly 100 but many different configurations and variations exist. Introducer sheath assembly 100 includes an introducer sheath 110 and a dilator 120 that may initially be assembled into introducer sheath 110. Dilator 120, which extends from a proximal end 121 to a tapered distal end 122, generally functions to facilitate insertion of introducer sheath 110 into the patient's vasculature. Introducer sheath 110 may have a shaft 111 that extends distally from a hub 112. When assembled, distal end 122 of dilator 120 extends distally from shaft 111 of introducer sheath 110. Handles 113 may extend from hub 112 to provide a gripping surface. In typical use cases, after introducer sheath 110 has been inserted into the patient's vasculature, dilator 120 will be withdrawn thereby allowing introducer sheath 110 to be used to insert a catheter or other device.

When an introducer sheath is used to introduce a catheter (or other device) into the patient's vasculature, it is common to employ fluoroscopy to confirm the position of the catheter and maneuver it as necessary into the desired location. For example, when angioplasty is performed, it is important that the catheter be positioned to align the balloon with the obstructed portion of the artery or vein. Given that fluoroscopy exposes the patient and clinicians to harmful radiation, it is generally desirable to minimize its use during a procedure. Yet, many procedures demand precise catheter placement that typically requires prolonged use of fluoroscopy. Furthermore, once the catheter is positioned, it is not uncommon for the catheter to move, which may require repeated use of fluoroscopy to reposition the catheter.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to an introducer sheath that includes a displacement sensor for detecting displacement of a catheter within the introducer sheath. The displacement sensor may employ induction-based, optical-based, mechanical-based or other techniques for detecting the displacement. The displacement sensor may include a display on which the detected displacement is presented to a clinician. The displacement sensor may allow the clinician to provide user input to reset the displacement.

In some embodiments, an introducer sheath may include a hub forming a proximal opening to a lumen, a shaft extending distally from the hub with the lumen extending through the shaft to form a distal opening and a displacement sensor. In some embodiments, the displacement sensor may include a sensor unit positioned to detect displacement of a catheter within the lumen and to output signals representing the detected displacement. In some embodiments, the displacement sensor may include a control unit that is configured to receive the signals from the sensor unit and to maintain a displacement value based on the signals. In some embodiments, the control unit may be configured to output the displacement value. In some embodiments, the displacement sensor may include a display on which the displacement value may be displayed.

In some embodiments, the sensor unit may be positioned at least partially around the shaft. In some embodiments, the sensor unit may include an inductive element, and the signals representing the detected displacement may represent variations in inductance. In some embodiments, the sensor unit may include one or more optical sensors, and the signals representing the detected displacement may represent light received by the one or more optical sensors. In some embodiments, the sensor unit may include one or more rollers, and the signals representing the detected displacement may represent rotation of the one or more rollers.

In some embodiments, the displacement sensor may include an input element, and the control unit may be configured to reset the displacement value when the input element is actuated. In some embodiments, the input element may be a display, and the displacement value may be displayed on the display. In some embodiments, the control unit may output an alarm in response to the displacement value being changed. In some embodiments, the displacement sensor may include wireless circuitry, and the displacement value may be transmitted via the wireless circuitry to an external device.

In some embodiments, an introducer sheath may include a hub forming a proximal opening to a lumen, a shaft extending distally from the hub with the lumen extending through the shaft to form a distal opening and a displacement sensor. In some embodiments, the displacement sensor may include a sensor unit positioned to detect displacement of a catheter within the lumen and to output signals representing the detected displacement. In some embodiments, the displacement sensor may include a control unit that is configured to receive the signals from the sensor unit and to maintain a displacement value based on the signals. In some embodiments, the displacement sensor may include a display on which the control unit displays the displacement value.

In some embodiments, the sensor unit may include an inductive element that is positioned adjacent the lumen, and the signals representing the detected displacement may represent variations in inductance of the inductive element caused when the catheter is displaced within the lumen. In some embodiments, the catheter may be positioned within another catheter when the catheter is displaced within the lumen.

In some embodiments, the sensor unit may include one or more optical sensors, and the signals representing the detected displacement may represent light reflected from markings on the catheter when the catheter is displaced within the lumen. In some embodiments, the sensor unit may include one or more rollers, and the signals representing the detected displacement may represent rotation of the one or more rollers caused by the catheter when the catheter is displaced within the lumen.

In some embodiments, the control unit may be configured to reset the displacement value in response to user input. In some embodiments, the user input may be received via the display or an input element.

In some embodiments, an introducer sheath may include a hub forming a proximal opening to a lumen, a shaft extending distally from the hub with the lumen extending through the shaft to form a distal opening and a displacement sensor integrated into the shaft and having a display. In some embodiments, the displacement sensor may be configured to calculate a displacement value as a catheter is displaced within the lumen and to display the displacement value on the display. In some embodiments, the displacement sensor may calculate the displacement value based on: changes in inductance caused by the catheter as the catheter is displaced within the lumen; light reflected by the catheter as the catheter is displaced within the lumen; or rotation caused by the catheter as the catheter is displaced within the lumen. In some embodiments, the displacement sensor may be configured to reset the displacement value in response to user input.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 illustrates an example of a prior art introducer sheath assembly;

FIG. 2 illustrates an example of an introducer sheath assembly having a displacement sensor, in accordance with some embodiments;

FIGS. 3A and 3B are cross-sectional views illustrating an introducer sheath having a displacement sensor, in accordance with some embodiments;

FIGS. 4A and 4B are cross-sectional views illustrating another introducer sheath having a displacement sensor, in accordance with some embodiments;

FIGS. 5A and 5B are cross-sectional views illustrating another introducer sheath having a displacement sensor, in accordance with some embodiments; and

FIGS. 6A-6D provide an example of how an introducer sheath having a displacement sensor may be employed, in accordance with some embodiments.

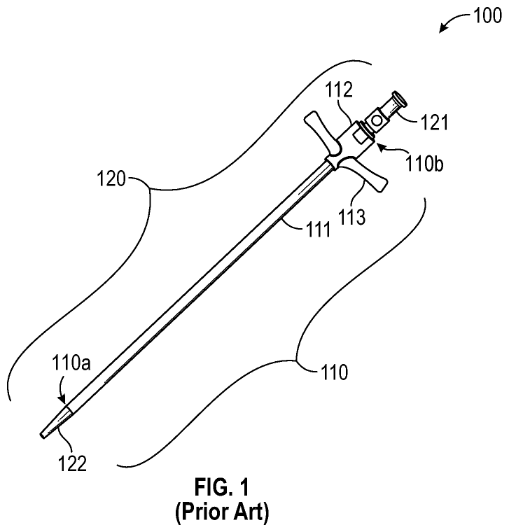

### DESCRIPTION OF EMBODIMENTS

Embodiments of the present disclosure extend to many different types and configurations of introducer sheaths, introducer sheath assemblies and intravenous catheter systems that include introducer sheaths. For example, any introducer sheath may integrate a displacement sensor in accordance with some embodiments.

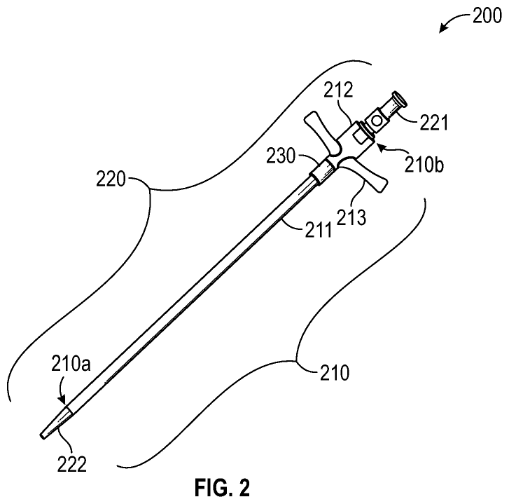

FIG. 2 illustrates one example of an introducer sheath assembly 200 having an introducer sheath 210 that integrates a displacement sensor 230 in accordance with some embodiments. Introducer sheath assembly 200 may have similar components as introducer sheath assembly 100 described in the background. For example, these components include introducer sheath 210 having a shaft 211 that extends to distal end 210a, a hub 212 positioned at proximal end 210b and handles 213 that extend from hub 212. The components may also include a dilator 220 having a proximal end 221 and a distal end 222. It is noted, however, that an introducer sheath configured in accordance with some embodiments need not include, incorporate or otherwise utilize a dilator or any other device. FIG. 2 should therefore be viewed as an example of how a displacement sensor 230 may be integrated into one of the many different types of introducer sheaths that may be available.

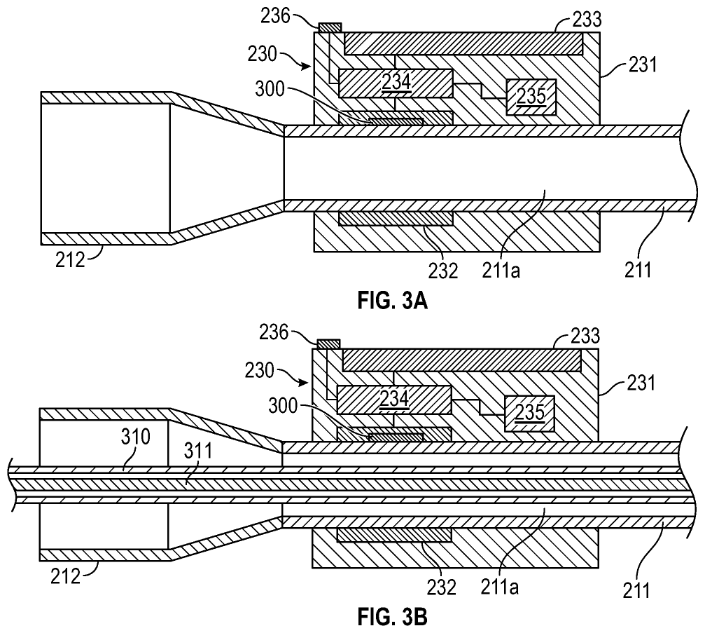

FIGS. 3A and 3B are partial cross-sectional side views illustrating one example of how displacement sensor 230 may be integrated into introducer sheath 210 as well as various components that displacement sensor 230 may include in some embodiments. FIG. 3A represents introducer sheath 210 when a catheter (or other device) has not yet been inserted through introducer sheath 210, whereas FIG. 3B represents introducer sheath 210 when a guide catheter 310 and a microcatheter 311 have being inserted through introducer sheath 210. In this context, a “guide catheter” should be construed as a catheter that may be used to guide the insertion of a microcatheter or other device such as a guide wire. Microcatheter 311 may therefore also be viewed as representing a guide wire or other device capable of being inserted through guide catheter 310. Also, even though FIG. 3B depicts an example where both a guide catheter 310 and microcatheter 311 are inserted into introducer sheath 210, it should not be viewed as limiting the depicted example to such use cases.

Displacement sensor 230 may include a housing 231 that is coupled to, integrated with or otherwise positioned along shaft 211 of introducer sheath 210. In the depicted example, housing 231 is positioned adjacent to hub 212, but may be positioned at other locations including on hub 212 or spaced distally from hub 212 in some embodiments. In FIGS. 3A and 3B, various components are shown as being positioned on or in housing 231. However, not all of the depicted components need to be included in displacement sensor 230 in some embodiments. In the depicted embodiment, displacement sensor 230 includes a sensor unit 232 that may be positioned at least partially around or adjacent to shaft 211 and may have an inductive element 300 (e.g., an electrical coil) that is positioned on, in or adjacent to a sidewall of shaft 211. Accordingly, FIGS. 3A and 3B represent embodiments where displacement sensor 230 is an induction-based displacement sensor. To enable induction-based displacement sensing, the catheter, microcatheter, guidewire, etc. having its displacement sensed can include metal.

Displacement sensor 230 may also include a control unit 234 that is electrically coupled to sensor unit 232. Control unit 234 may be configured to receive signals from sensor unit 232 and, based on such signals, detect displacement of a catheter or other device within lumen 211a of shaft 211. For example, either or both guide catheter 310 and microcatheter 311 may include elements that modify the inductance of inductive element 300 as the elements move past inductive element 300. In some embodiments, these elements may be in the form of metal (e.g., nickel titanium, or Nitinol, wire) embedded into guide catheter 310, microcatheter 311 or any other catheter that may be compatible with introducer sheath 210. Control unit 234 may be configured to detect variations in the inductance of inductive element 300, or receive signals generated by sensor unit 232 that represent such variations, to thereby detect how far and in which direction guide catheter 310, microcatheter 311 or another catheter has been displaced within shaft 211. Control unit 234 may also be configured to store one or more values representing the detected displacement (or “displacement value”).

In some embodiments, displacement sensor 230 may include a display 233 that may be positioned at an exterior surface of housing 231 so that display 233 may be visible during use of introducer sheath 210. For example, display 233 may be oriented in an upward direction when introducer sheath 210 is inserted into the patient's vasculature. Any type of display, such as LED or LCD, may be employed. Display 233 may be electrically coupled to control unit 234 and may receive display signals containing information representing the current displacement value. In other words, control unit 234 may cause the displacement value to be displayed on display 233. Control unit 234 may also cause other information to be displayed on display 233 such as, for example, an insertion velocity, a timer, etc.

In some embodiments, displacement sensor 230 may include wireless circuitry 235 (e.g., Bluetooth or Wi-fi circuitry) which control unit 234 may employ to wirelessly transmit the displacement value or other information to one or more other systems (e.g., to display the displacement on augmented reality glasses that a clinician is wearing during a procedure, to a remote display being monitored by a clinician, to a storage system, etc.). In some embodiments, wireless circuitry 235 may also enable control unit 234 to receive communications from one or more other systems. In some embodiments, such communications may include communications defining characteristics of a catheter or other device whose displacement is to be detected (e.g., the distance at which the elements are spaced), communications defining a mode of operation, communications providing updated firmware, etc. Although not shown, in some embodiments, displacement sensor 230 may alternatively or additionally include circuitry for sending or receiving communications/information over a wired connection. In some embodiments, displacement sensor 230 may include an input element (or elements) 236 (e.g., a button, a switch, a sensor, a touch screen, etc.) for providing manual user input to control unit 234. In some embodiments, input element 236 may function to enable a user to reset (or set) the displacement value. In some embodiments, input element 236 may be incorporated into display 233 (e.g., when display 233 is a touch screen). Although not shown, displacement sensor 230 may also include a power source (e.g., a battery) for powering the various components.

As suggested above, when introducer sheath 210 includes displacement sensor 230 having inductive element 300, the displacement of any catheter may be detected as it passes through shaft 211 if the catheter includes elements that alter the inductance of inductive element 300 (e.g., metal elements that react to a magnetic field created by inductive element 300). For example, if guide catheter 310 includes elements spaced at (or patterned with) 1 cm increments, control unit 234 may be configured to increment or decrement the displacement value for guide catheter 310 by 1 cm each time control unit 234 detects a change in inductance indicative of an element passing by inductive element 300. Whether control unit 234 increments or decrements the displacement value may be based on a known profile of the changed inductance. In other words, control unit 234 may detect from the profile of the changed inductance whether guide catheter 310 is being inserted into or withdrawn from shaft 211. Furthermore, using the known profile, control unit 234 may detect displacement at a high level of granularity (e.g., mm increments).

As it tracks/calculates this displacement, control unit 234 may output display signals to display 233 to cause the amount of displacement to be displayed to the user. Accordingly, assuming the displacement value is set to 0 before guide catheter 310 is inserted into introducer sheath 210 and that the user desires to insert the guide catheter to a depth of 10 cm, the user may watch display 233 while inserting guide catheter 310 and stop inserting guide catheter 310 once display 233 reflects a displacement value of 10 cm. A similar process may be employed when inserting microcatheter 311 through guide catheter 310 or when inserting any other compatible catheter.

In some embodiments, to facilitate relative positioning of a catheter, the user may actuate input element 236 to zero (or reset) the displacement value and then further insert the catheter or insert another catheter. For example, after inserting guide catheter 310 to a desired depth, the user may actuate input element 236 to zero the displacement value and may then insert microcatheter 311 through guide catheter 310. Based on the signals received from sensor unit 232 (e.g., signals indicative of changes in the inductance of inductive element 300) as microcatheter 311 is inserted, control unit 234 may detect the displacement of microcatheter 311 and cause display 233 to be updated accordingly. The user may proceed to insert microcatheter 311 until display 233 indicates that microcatheter 311 has been inserted to a depth of 10 cm (i.e., to the same depth as guide catheter 310). At this point, the user may again zero the displacement and then further insert microcatheter 311 to a desired depth relative to the insertion depth of guide catheter 310. In this scenario, the detected displacement that control unit 234 causes to be displayed will represent the displacement of microcatheter 311 relative to guide catheter 310.

Once microcatheter 311 (or any other compatible catheter) has been inserted to the desired depth (e.g., when microcatheter 311 is positioned at the site where a procedure is to be performed), microcatheter 311 may be secured and input element 236 may be actuated to zero the displacement value. This zeroed displacement value can represent that microcatheter 311 is in the desired location. If control unit 234 subsequently detects any displacement of microcatheter 311, control unit 234 may update the displacement value and cause display 233 to be updated to reflect the change in displacement, which in turn may immediately represent to the user that microcatheter 311 has moved. In this way, the user may easily detect when microcatheter 311 has moved from the desired location.

In some embodiments, control unit 234 may provide a mode of operation in which it will output an alarm when it detects displacement of a catheter or other device within introducer sheath 210. For example, displacement sensor 230 could include an input element 236 that allows a user to enter such a mode after a catheter has been inserted to a desired depth. In some embodiments, as part of entering this mode, control unit 234 may also zero the displacement value. Once in this mode, if control unit 234 detects any displacement, or possibly any displacement that exceeds a defined threshold, it may output an alarm to alert a clinician that the catheter may need to be repositioned. In some embodiments, control unit 234 may be configured to enter this mode automatically, such as after it has failed to detect further displacement of a catheter over a defined amount of time.

The above-described embodiments that employ inductive element 300 to sense displacement of a catheter may provide a number of benefits. For example, inductive element 300 need not be positioned in lumen 211a and may therefore be isolated from any fluid (e.g., blood) that may be contained in lumen 211a. For similar reasons, inductive element 300 may be capable of detecting displacement of catheters having a variety of gauges and may be capable of detecting displacement of one catheter (or other device) that is passing through another catheter.

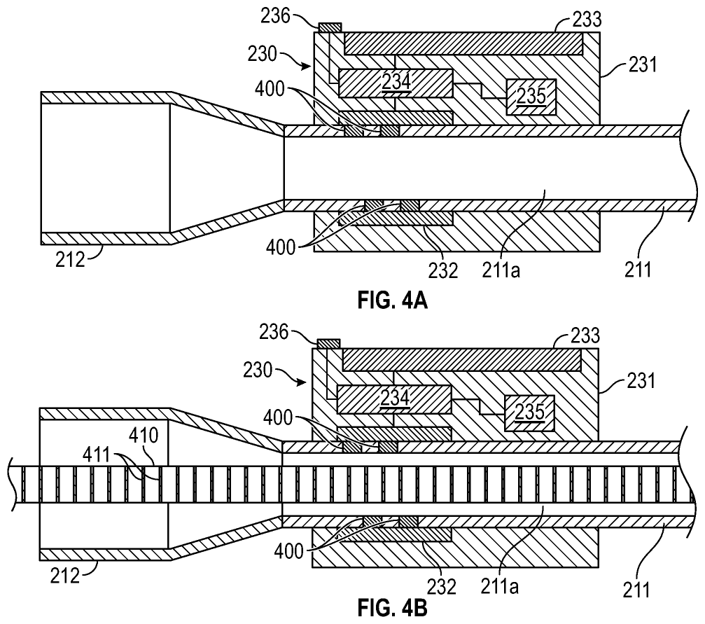

FIGS. 4A and 4B are partial cross-sectional side views illustrating another example of how displacement sensor 230 may be integrated into introducer sheath 210. In contrast to the induction-based displacement sensor 230 of FIGS. 3A and 3B, FIGS. 4A and 4B represent embodiments where displacement sensor 230 is optical-based. For example, sensor unit 232 may include one or more optical sensors 400 that may be integrated into shaft 211 or otherwise positioned to enable each optical sensor 400 to detect markings on a catheter or other device that is passed through introducer sheath 210. In the depicted example, sensor unit 232 includes a first set of two optical sensors 400 positioned on one side of shaft 211 and a second set of two optical sensors 400 that are positioned on an opposite side of shaft 211 and offset relative to the first set of two optical sensors 400. However, in other embodiments, any number and arrangement of optical sensors 400 could be employed. In some embodiments, by including multiple sets of optical sensors 400 that are offset from one another, displacement sensor 230 may be able to detect the position of a catheter with a high level of granularity. FIGS. 4A and 4B represent that an optical-based displacement sensor 230 may include similar components as described above. Therefore, a repeated description of such components is not provided.

FIG. 4B provides an example of how a catheter 410 may be configured to be compatible with introducer sheath 210 when it has an optical-based displacement sensor 230. As shown, catheter 410 may include markings 411 that are spaced along the length of catheter 410. Markings 411 may represent any type of indicia that is capable of being detected by optical sensor(s) 400. As an example only, markings 411 may be formed by printing lines radially around the outer surface of catheter 410 at a fixed interval where such markings 411 may be configured to reflect light emitted by optical sensors 400. In some embodiments, control unit 234 may be preprogrammed with or may receive user input identifying the spacing between markings 411.

Control unit 234 may be configured to receive signals from each optical sensor 400 which identify when the optical sensor detects a marking 411. Such signals may be output directly to control unit 234 or may be handled by intermediate circuitry. In any case, control unit 234 may be configured to process the signals in a similar manner as described above to determine how far and in which direction catheter 410 is being displaced.

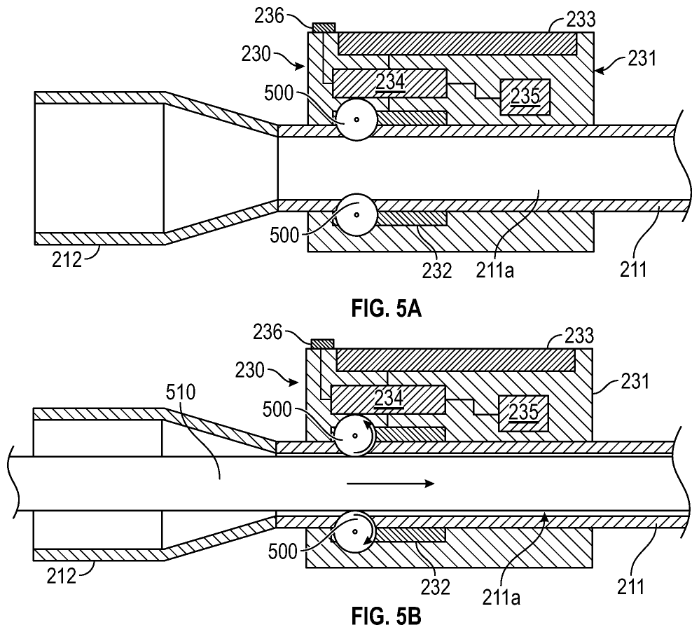

FIGS. 5A and 5B are partial cross-sectional side views illustrating another example of how displacement sensor 230 may be integrated into introducer sheath 210. In this example, displacement sensor 230 is mechanical-based. For example, FIG. 5A shows that sensor unit 232 may include one or more rollers 500 that extend into lumen 211a of shaft 211. Rollers 500 may be configured to contact the outer surface of a catheter (or other device) and rotate (or roll) as the catheter is moved within lumen 211a. In some embodiments, two rollers 500 positioned on opposing sides of shaft 211 may be employed. In some embodiments, rollers 500 may be configured to move inwardly and outwardly to accommodate catheters or other devices of different gauges. For example, rollers 500 may be coupled to sensor unit 232 in a manner that biases rollers 500 into lumen 211a while allowing rollers 500 to move outwardly (e.g., by pivoting distally or proximally, in an axial direction, etc.) when a catheter is inserted between rollers 500. Sensor unit 232 may include circuitry (e.g., one or more rotary encoders) which outputs signals representing how far and in which direction rollers 500 have rotated. Control unit 234 may be configured to process the signals in a similar manner as described above to determine how far and in which direction a catheter 510 is being displaced. Notably, in embodiments where introducer sheath 210 includes a mechanical-based displacement sensor 230, the displacement of virtually any catheter may be detected. In some embodiments, rollers 500 may be textured to prevent slippage such as when there is blood on the catheter.

As evidenced by these examples, displacement sensor 230 may employ various types of sensor units 232 including, but not limited to, inductance-based, optical-based and mechanical-based sensor units 232. Regardless of the type of sensor unit 232, displacement sensor 230 may be configured to detect and display in real-time the displacement of a catheter or other device as it moves within lumen 211a of shaft 211. Accordingly, by integrating displacement sensor 230 into introducer sheath 210, a clinician may monitor the position or depth of a catheter or other device as it is inserted into or moved within a patient's vasculature. In some embodiments, this monitoring may be performed with high precision and may reduce or eliminate the need to employ fluoroscopy during a procedure.

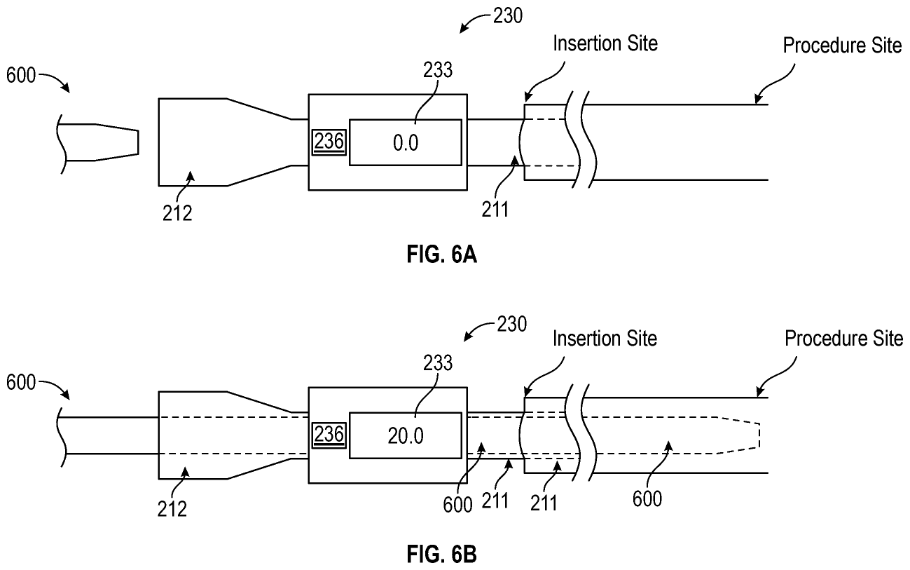

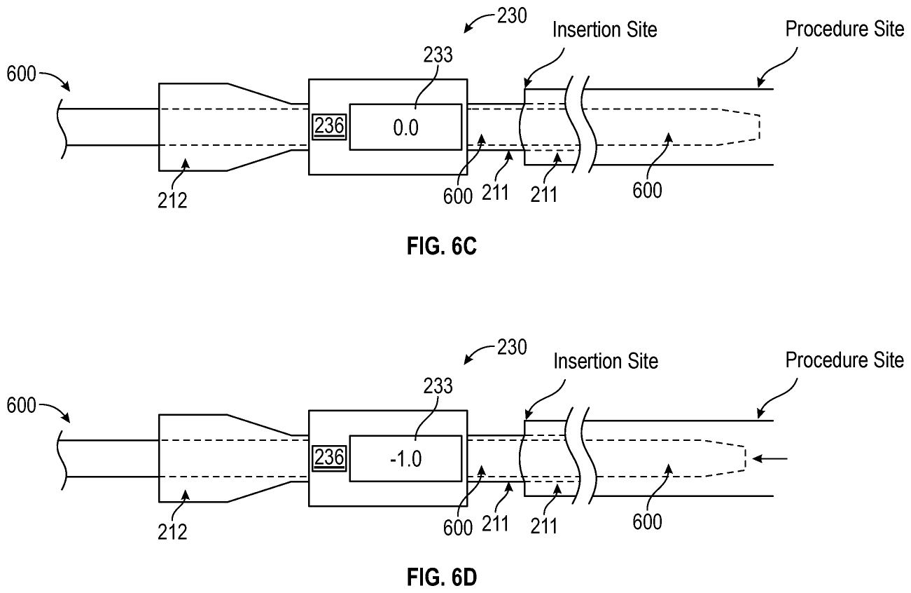

FIGS. 6A-6D provide an example of how displacement sensor 230, which may be configured in any of the various ways described above, may be used when a catheter 600 is inserted into a patient's vasculature via introducer sheath 210. In this example, there is assumed to be a procedure site at which the tip of catheter 600 should be positioned.

In FIG. 6A, introducer sheath 210 has been inserted into the patient's vasculature at an insertion site but catheter 600 has not yet been inserted into introducer sheath 210. It is assumed that displacement sensor 230 is currently reporting a displacement value of zero as represented by 0.0 being displayed on display 233. As examples only, a clinician could have pressed input element 236 (or utilized a touch screen interface of display 233), provided input via wireless (or wired) circuitry 235, powered on displacement sensor 230, etc. to cause control unit 234 to reset the displacement value to zero. It is noted, however, that the displacement value need not be set to zero or any particular value prior to inserting a catheter. Notably, in some embodiments, a clinician may insert a catheter up to the insertion site and then zero the displacement value so that the displacement value will define a depth relative to the insertion site.

Turning to FIG. 6B, it is assumed that the clinician has inserted catheter 600 through introducer sheath 210 and into the patient's vasculature until its distal end is positioned at the procedure site. It is also assumed that the depth of insertion relative to displacement sensor 230 is 20 cm as represented by 20.0 being displayed on display 233. In some embodiments, the clinician could have known that the procedure site was at a depth of 20 cm (e.g., based on prior use of displacement sensor 230). In such embodiments, the clinician could have positioned catheter 600 at this depth by monitoring display 233 while inserting catheter 600 and ceasing to insert catheter 600 once the display read 20.0. In other embodiments, such as when the depth of the procedure site is not known, any suitable technique (e.g., fluoroscopy) could be used to guide the insertion of catheter 600 to the proper depth. In any case, as catheter 600 passes through displacement sensor 230, control unit 234 may employ signals received from sensor unit 232 to track/calculate the depth to which catheter 600 is currently inserted using any of the above-described or other suitable techniques, update the displacement value accordingly and cause display 233 to reflect the current displacement value.

Turning to FIG. 6C, with catheter 600 positioned at the procedure site, it is assumed that the clinician has provided input to cause the displacement value to be reset to zero. For example, the clinician may secure catheter 600 to the patient's skin to prevent it from moving and may then press input element 236 to cause control unit 234 to reset the displacement value to zero. Then, as represented in FIG. 6D, it is assumed that catheter 600 moves proximally by 1 cm. For example, the patient could have pulled on, bumped or otherwise interacted with catheter 600 to cause this movement. As catheter 600 moves, control unit 234 may receive signals from sensor unit 232 indicative of this movement and may update the displacement value accordingly and cause the updated displacement value, which in the example is −1.0, to be displayed. As a result, the clinician could immediately determine that catheter 600 has moved by viewing display 233. Also, in some embodiments, control unit 234 could have caused an alarm to be output upon detecting this movement. After detecting this movement, the clinician could return catheter 600 to the procedure site by inserting it until display 233 again reads 0.0.

From the example of FIGS. 6A-6D, it can be seen that displacement sensor 230 may enable a clinician to detect and record the depth of a procedure site (or other position) within the patient's vasculature so that a catheter (or other device) could subsequently be returned to the same procedure site with precision. For example, the clinician could perform the process represented in FIG. 6B to determine that the procedure site is at a depth of 20 cm. After catheter 600 is removed, it if were again desired to insert a catheter to the procedure site, displacement sensor 230 could be used during the insertion to know precisely when the catheter has reached a depth of 20 cm. A similar technique could be employed to position a catheter at a particular depth relative to another position. For example, if the depth of the hepatic artery relative to an insertion site is known, and the clinician desires to insert a microcatheter a specified depth into the hepatic artery, the above-described techniques could be employed to locate the hepatic artery and then track further insertion of the microcatheter to the specified depth.

Displacement sensor 230 may also be employed to measure distances intravascularly. For example, upon positioned a catheter at a first location within the vasculature, a clinician may reset the displacement value and then advance the catheter to second location. Upon reaching the second location, the displacement value presented on display 233 may define the distance between the first and second locations.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. An introducer sheath, comprising:
a hub forming a proximal opening to a lumen;
a shaft extending distally from the hub, the lumen extending through the shaft to form a distal opening; and
a displacement sensor, comprising:
a sensor unit positioned to detect displacement of a catheter within the lumen and to output signals representing the detected displacement; and
a control unit that is configured to receive the signals from the sensor unit and to maintain a displacement value based on the signals, wherein the control unit is configured to output the displacement value.

2. The introducer sheath of claim 1, wherein the displacement sensor further comprises a display, and wherein outputting the displacement value comprises causing the displacement value to be displayed on the display.

3. The introducer sheath of claim 1, wherein the catheter comprises a guide catheter and a microcatheter or guidewire.

4. The introducer sheath of claim 1, wherein the sensor unit includes an inductive element, and wherein the signals representing the detected displacement represent variations in inductance.

5. The introducer sheath of claim 1, wherein the sensor unit includes one or more optical sensors, and wherein the signals representing the detected displacement represent light received by the one or more optical sensors.

6. The introducer sheath of claim 1, wherein the sensor unit includes one or more rollers, and wherein the signals representing the detected displacement represent rotation of the one or more rollers.

7. The introducer sheath of claim 1, wherein the displacement sensor comprises an input element, and wherein the control unit is configured to reset the displacement value when the input element is actuated.

8. The introducer sheath of claim 7, wherein the input element is a display, and wherein outputting the displacement value comprises causing the displacement value to be displayed on the display.

9. The introducer sheath of claim 1, wherein the control unit is further configured to output an alarm in response to the displacement value being changed.

10. The introducer sheath of claim 1, wherein the displacement sensor further comprises wireless circuitry, and wherein outputting the displacement value comprises causing the displacement value to be transmitted via the wireless circuitry to an external device.

11. An introducer sheath comprising:
a hub forming a proximal opening to a lumen;
a shaft extending distally from the hub, the lumen extending through the shaft to form a distal opening; and
a displacement sensor comprising:
a sensor unit positioned to detect displacement of a catheter within the lumen and to output signals representing the detected displacement;
a control unit that is configured to receive the signals from the sensor unit and to maintain a displacement value based on the signals; and
a display on which the control unit displays the displacement value.

12. The introducer sheath of claim 11, wherein the sensor unit includes an inductive element that is positioned adjacent the lumen, wherein the signals representing the detected displacement represent variations in inductance of the inductive element caused when the catheter is displaced within the lumen.

13. The introducer sheath of claim 12, wherein the catheter is positioned within another catheter when the catheter is displaced within the lumen.

14. The introducer sheath of claim 11, wherein the sensor unit includes one or more optical sensors, and wherein the signals representing the detected displacement represent light reflected from markings on the catheter when the catheter is displaced within the lumen.

15. The introducer sheath of claim 11, wherein the sensor unit includes one or more rollers, and wherein the signals representing the detected displacement represent rotation of the one or more rollers caused by the catheter when the catheter is displaced within the lumen.

16. The introducer sheath of claim 11, wherein the control unit is configured to reset the displacement value in response to user input.

17. The introducer sheath of claim 16, wherein the user input is received via the display or an input element.

18. An introducer sheath comprising:
a hub forming a proximal opening to a lumen;
a shaft extending distally from the hub, the lumen extending through the shaft to form a distal opening; and
a displacement sensor integrated into the shaft and having a display, the displacement sensor being configured to calculate a displacement value as a catheter is displaced within the lumen and to display the displacement value on the display.

19. The introducer sheath of claim 18, wherein the displacement sensor calculates the displacement value based on one of:
changes in inductance caused by the catheter as the catheter is displaced within the lumen;
light reflected by the catheter as the catheter is displaced within the lumen; or rotation caused by the catheter as the catheter is displaced within the lumen.

20. The introducer sheath of claim 18, wherein the displacement sensor is configured to reset the displacement value in response to user input.
